(19) United States
(12) Patent Application Publication
(10) Pub. No.: US 2020/0082543 A1
(43) Pub. Date: Mar. 12, 2020

(54) ORAL CLEANING DETECTION SYSTEM

(71) Applicant: Che-Min Lin, Taipei City (TW)

(72) Inventor: Che-Min Lin, Taipei City (TW)

(21) Appl. No.: 16/122,895

(22) Filed: Sep. 6, 2018

(51) Int. Cl. G06T 7/20; G06Q 30/02; G06K 9/00; A61B 5/00; A61B 5/11; G09B 19/00

(52) U.S. Cl. CPC G06T 7/20; G06Q 30/0207; G06K 9/00758; A61B 5/0077; A61B 5/1123; G06F 8/60; A61B 5/4547; A61B 5/486; A61B 5/6898; G09B 19/0084; A61B 5/1128

(57) ABSTRACT

An oral cleaning detection system includes a remote data service platform and mobile smart device. The remote data service platform is built on a network platform including a tooth cleaning software module having a motion detection unit and reward unit; the mobile smart device may enter the remote data service platform to download the tooth cleaning software module, a photography equipment of the mobile smart device is turned on through the software to capture a user's teeth brushing motion, a motion track comparison is carried out with the tooth cleaning software module to judge whether a preset brushing motion is completed correctly, and at the same time, a user may be rewarded as an encouragement.

### TECHNICAL FIELD OF THE INVENTION

The present invention relates to an oral cleaning detection system, and more particularly to an oral cleaning system, detecting teeth brushing action and further maintaining effective teeth health by accumulating rewards through a reward mode.

### DESCRIPTION OF THE PRIOR ART

Good oral hygiene habits can prevent oral diseases from happening, and oral diseases relates closely to oral hygiene habits for sure. For example, periodontal disease is an oral disease mainly caused from bacterial invasion of periodontal tissue; if lighter, it may have odor in the mouth, redness and swelling of the gums, and if severe, teeth may shake or even fall off. Therefore, how to develop correct teeth brushing motion to maintain good oral hygiene is a very important issue. However, many people (especially children) still do not use toothbrushes correctly according to researches, including postures and time. Developing a correct and complete teeth brushing habit may be a well-known truth, but it's a difficult thing to practice one's daily practice. Although there are some auxiliary tools for cleaning teeth, and some people use music entertainment devices or timers to motivate users to follow the rules, the effects are not so remarkable as expected such that many people usually have oral problems caused from incorrect teeth brushing habits.

### SUMMARY OF THE INVENTION

To overcome the above shortcomings, the present invention is proposed.

The object of the present invention is to provide an oral cleaning detection system, capable of detecting a teeth brushing motion and accumulating rewards through a reward mode, thereby achieving effective detail hygiene maintenance.

To achieve the object mentioned above, the present invention proposes an oral cleaning detection system, including a remote data service platform and mobile smart device, the remote data service platform built on a network platform and including a downloadable tooth cleaning software module having a motion detection unit and reward unit; the motion detection unit detecting whether brushing motion and time are correct through a motion track, generating a detection signal through a comparison of the motion track, judging whether a preset brushing motion is completed correctly, and emitting a correct or wrong evaluation signal to the reward unit according to a comparison result; the reward unit giving a preset reward according to the correct or wrong evaluation signal; the mobile smart device referring to a tablet or smart phone having a networking function, the mobile smart device entering the remote data service platform to download the tooth cleaning software module, a photography equipment of the mobile smart device turned on through the tooth cleaning software module to capture a user's brushing motion, a motion track comparison carried out to judge whether the preset brushing motion is completed correctly.

According to the system mentioned above, the reward unit rewards the user with a preset amount of virtual currency if the brushing motion is correct.

### BRIEF DESCRIPTION OF THE DRAWINGS

FIG. 1 is a simplified block diagram of a preferred embodiment of the present invention; and

FIG. 2 is a schematic view of the embodiment of the present invention upon implementation.

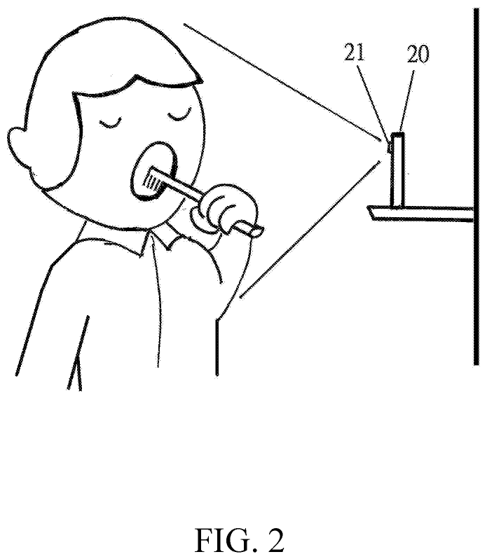

### DETAILED DESCRIPTION OF THE PREFERRED EMBODIMENTS

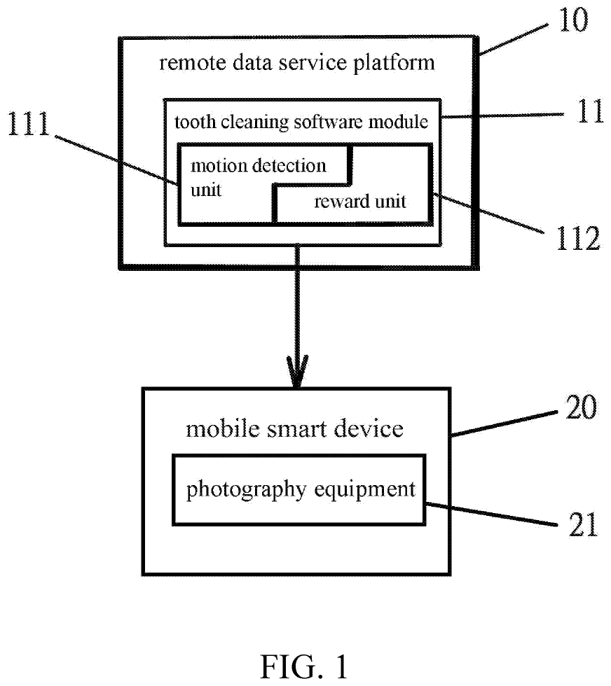

Referring to FIG. 1, which is a preferred embodiment of a simplified block diagram of the present invention includes a remote data service platform 10 built on the network platform and mobile smart device 20.

The remote data service platform 10 is built on a network platform; it includes a downloadable tooth cleaning software module 11 further including an motion detection unit 111 and reward unit 112, where the motion detection unit 111 can detect whether teeth brushing motion and time are correct through a motion track, generate a detection signal, and study the teeth brushing motion signal and then compare it with a preset motion track, thereby judging whether the preset teeth brushing motion is completed correctly. Thereafter, the motion detection unit 111 emits a correct or wrong evaluation signal to the reward unit 112, and the reward unit 112 then gives an output display according the correct or wrong evaluation signal. It is worth to mention that the remote data service platform 10 may have components such as a member identification module, tooth brushing guide module and memory module optionally besides the teeth cleaning software module 11. But, since these components are not the characteristics of the present invention, the details thereof are omitted here.

The mobile smart device 20 refers to a tablet or smart phone with a networking function. The mobile smart device 20 can be connected to the Internet and then enter the remote data service platform 10 to download the tooth cleaning software module 11. Thereafter, the mobile smart device 20 can turn on photography equipment 21 of the mobile smart device 20 to capture a user's teeth brushing motion through the tooth cleaning software module 11 of the remote data service platform 10, and the motion detection unit 111 of the tooth cleaning software module 11 carries out a comparison through a motion track to judge whether it completes the teeth brushing motion correctly and emits a correct or wrong evaluation signal to the reward unit 111; if it is correct, the reward unit 112 awards a prize to the user.

The reward unit 112 of the tooth cleaning software module 11 of the remote data service platform 10 uses a reward as an incentive to encourage users to develop correct teeth brushing postures; such kind of reward way may issue the appropriate amount of virtual currency. Since the virtual currency is high in safety and difficult to be stolen or tampered, it has a very high reward value. In addition, of course, a way of rewarding with the member points can be chosen, and the points are redeemed according to the number of points.

The mode of the present invention is an interactive system adapted to generate a correct or wrong teeth cleaning motion signal through the comparison of a motion track and encourage users to develop a correct teeth brushing motion, using the positive encouragement represented by the reward to develop a right behavior mode. Those with ordinary knowledge in the field should understand that users' brushing motion should be captured within the appropriate range when the photography equipment 21 of the mobile smart device 20 is turned on, and the tooth cleaning software module 11 is also provided with a calibration function. In addition, the wrong evaluation signals are generated unceasingly after the comparisons of the motion detection unit 111 of the tooth cleaning software module 11, which means that a user's brushing motion is incorrect, and this information may also be received by a second person or professional medical institution to carry out further counseling.

The present invention provides a smart interactive teeth brushing system adapted to evaluate whether a brushing motion is correct or not, mainly hoping to develop a correct brushing motion to ensure a better oral hygiene habit, thereby reducing the chance of oral disease, and achieving the purpose of maintaining dental health.

The above description exemplifies a preferred embodiment of the present invention and how to implement the present invention. However, the examples, embodiments, or instruction semantics should not be construed as limiting the present invention. With the specific implementation of the aforementioned technical means, the present invention not only is very convenient to use, but also accumulates rewards by detecting brushing motions, thereby effectively maintaining dental health.

I claim:

1. An oral cleaning detection system, comprising a remote data service platform and mobile smart device, said remote data service platform built on a network platform and comprising a downloadable tooth cleaning software module having a motion detection unit and reward unit; said motion detection unit detecting whether brushing motion and time are correct through a motion track, generating a detection signal through a comparison of said motion track, judging whether a preset brushing motion is completed correctly, and emitting a correct or wrong evaluation signal to said reward unit according to a comparison result; said reward unit giving a preset reward according to said correct or wrong evaluation signal; said mobile smart device referring to a tablet or smart phone having a networking function, said mobile smart device entering said remote data service platform to download said tooth cleaning software module, a photography equipment of said mobile smart device turned on through said tooth cleaning software module to capture a user's brushing motion, a motion track comparison carried out with said motion detection unit to judge whether said preset brushing motion is completed correctly.

2. The system according to claim 1, wherein said reward unit rewards said user with a preset amount of virtual currency if said brushing motion is correct.
